(19) United States
(12) Patent Application Publication
(10) Pub. No.: US 2026/0306161 A1
(43) Pub. Date: Oct. 8, 2026

(54) A RAPIDLY DEPLOYABLE BIOMEDICAL EMF EMITTING DEVICE AND METHODS OF USE

(71) Applicants: Matthew Maale, Dallas, TX (US); Gerhard Maale, Plano, TX (US); Gregory Tobin, Frederick, MD (US); Stephen Dollery, South Riding, VA (US)

(72) Inventors: Matthew Maale, Dallas, TX (US); Gerhard Maale, Plano, TX (US); Gregory Tobin, Frederick, MD (US); Stephen Dollery, South Riding, VA (US)

(21) Appl. No.: 19/478,932

(22) PCT Filed: Apr. 29, 2024
(86) PCT No.: PCT/US2024/026743

§ 371 Date: Oct. 27, 2025

Related U.S. Application Data
(60) Provisional application No. 63/498,992, filed on Apr. 28, 2023.

(51) Int. Cl. A61B 18/12; A61B 18/00; A61B 18/14; A61F 2/46

(52) U.S. Cl. CPC A61B 18/1206; A61B 18/14; A61F 2/46; A61B 2018/00005; A61B 2018/00178; A61B 2018/1465; A61F 2002/465

(57) ABSTRACT

EMF emitting devices and methods of using EMF emitting devices that apply one or more EMFs to one or more objects to generate heat due to electrical resistance of an induced current. Specifically, EMF emitting devices and methods are used for rendering nearby biological agents more susceptible to the immune system and/or antimicrobial agents or for softening bone cement proximate to medical implants.

### TECHNICAL FIELD

The present invention relates to devices and methods for applying treatment of one or more customizable electromagnetic field (EMF) focal points via superposition of an EMF to generate heat due to electrical resistance from an induced current on implanted metal objects within a patient's body or bone cement. The heat generated by the device of the present invention or other heat generating devices is effective in softening bone cement proximate to medical implants and in rendering nearby infectious microbes more susceptible to antimicrobials and/or the surrounding environment. The present invention also relates to a novel bone cement which reacts to EMFs by being impregnated with EMF responsive materials for the purpose of biological treatments or rendering the bone cement plastic.

### BACKGROUND

Medical implants are used in a variety of applications including surgical repairs, bone and joint replacements, supplementing physiological actions, and the like. Some implants are often affixed to a location in the body via a polymer of some type, such as bone cement, which may be made with poly(methyl methacrylate) (PMMA) or similar materials. Although most patients experience years of trouble-free use with implanted devices, some implants fail and require removal and/or replacement. The removal of medical implants or other affixed materials, especially those cemented into bone, can be difficult. Often the implant is sawed off and a considerable amount of bone is lost due to the extraction process, making any subsequent revisions more difficult and amputation probability increased. Procedures of this type also produce debris which must be removed from the surgical site causing additional time and effort. In addition, fragmentation of the implant, cement, and bone can exacerbate the injury due to the potential introduction of materials and debris into the surgical site, resulting in infections and future complications. From the surgeon's point of view, implant removal is a difficult and laborious process, and the use of a high-speed saw can cause hearing damage.

Infections can cause serious disease and death. Many types of pathogens, especially bacteria and fungi, can form biofilms which create a physical barrier that reduces the effectiveness of antimicrobial agents, such as antibiotics, and/or the immune system. In addition, many pathogens have developed genetic resistance mechanisms to antimicrobials. Today, the majority of infections seen in surgical implantations form biofilms that are resistant to front-line antibiotic treatments. Approximately 3% of patients with surgical implants develop bacterial or fungal infections which respond poorly, if at all, to aggressive intravenous antibiotic treatment. In many cases, the infections are serious enough to warrant removal, debridement, and even replacement of the implant. As a result, there is a need to provide a non-invasive solution for treating infections occurring in or around medical implants.

The mechanism of induction heaters and induction power systems is well known. The advantage of being able to wirelessly transfer power via an EMF has proven to be extremely valuable in many industries, especially when the EMF passes through other materials without affecting them. One of the first patents on induction heaters, U.S. Pat. No. 2,181,274, plus a large body of other patents have been developed from this technology for specific purposes. More recent patents discuss in detail the inner workings of induction power systems and coils, such as U.S. Pat. No. 8,803,649. To date, these systems have rarely been used in the biomedical world unless used for wireless powering. Heating materials in the body, especially rapid heating, is a novelty that has not been explored.

For an induction power system to transfer power to an object, an EMF must be generated by an electrically conducting wire or pipe, often conducting AC, especially when used to generate heat. EMFs are comprised of vectors. When vectors are superimposed on top of each other, a resultant vector can have a greater magnitude in a new direction. It is also known that EMFs create currents on the surface level of the metal to some thickness depth that they are heating, and that alternating EMFs and their eddy currents generate resistance and become an effective heating mechanism which can heat objects substantially and quickly. Induction heaters facilitate rapid heating which is beneficial for generating heat spikes and a rapid heating curve. If directed correctly, an EMF can make heat spikes which are focused to affect only the desired areas. However, the current designs of most EMF systems have significant limitations. Historically speaking, most induction systems which are designed to carry high power and generate strong EMFs use a copper coil which is rigid in nature while others that are flexible are limited to carrying lower power. In addition, such induction systems are not designed to rapidly connect and disconnect from their power and control systems with ease or be exchanged for different diameter and length wires for various applications. Existing devices are also not designed to create custom EMFs or focal points. Existing devices are also not designed to operate with wire systems that have inconsistent field strengths, shape, diameter, distance to target, and other intricacies that may be found in a hospital operating room or treatment environment.

Most EMF AC power systems are not designed to have an EMF field that is modified by multiple different power variable techniques and typically utilize just one system as the main control. Most power systems are designed to simply increase and optimize power transfer. This system is designed to be able to optimize these characteristics and modulate the fields effective range.

It would be desirable to have an induction system which comprises a wire system in which an EMF can be created and adjusted easily by a physician, nurse, or technician to have a desired focal point on a body part of a patient. In addition, it would be desirable to have the wire system cooled so as to not burn or damage surrounding materials or the patient as the conductive wire generates heat due to resistance. It would also be desirable to be able to create a custom local focal point of high EMF in a target area. It would also be desirable to provide shielding in portions of the wire so as not to heat other nearby objects or structures. It would also be desirable to be able to have a semi-rigid wire which can conform to and retain a desired shape. It would be desirable to have rapidly deployable orientation guides. It would also be desirable to be able to easily interchange lengths and diameters of the conducting wire for different treatment applications. It would be desirable to have a power system capable of increasing the EMF by increasing the voltage or adjusting the oscillation to match the resonant frequency, depending on the orientation of the wire system. It would be desirable to have a means to heat bone cement without the presence of a nearby metal object, so as to render it pliable to be removed during surgery, or to heat it in the case of rendering a biological agent susceptible to antibiotics. Lastly, it would also be desirable to have a power system that can adapt to these new orientations, which is quite adjustable manually and/or with the preprogrammed controls.

### SUMMARY OF THE INVENTION

The present invention discloses EMF emitting devices and methods of using one or more EMF emitting devices that apply one or more EMFs to one or more objects to generate heat due to electrical resistance of an induced current. One purpose of the heat is to alter a nearby material and/or biological properties of surrounding materials. One method uses this heat for the purpose of rendering nearby biological agents more susceptible to the immune system and/or antimicrobial agents. This has been shown experimentally by using metal coupons onto which a biofilm was grown and heating the coupons by means of an EMF in solutions with and without antibiotics present and with a control which has antibiotics present. The application of EMFs to heat the coupons shows the biofilm dying at elevated temperatures, dying at lower elevated temperatures with the presence of antibiotics, and not dying at body temperature with antibiotics present. This thus demonstrates a combined therapy and its effectiveness, showing other treatments limitations or ineffectiveness. In another method, the applied heat has been shown experimentally to cause a temperature-sensitive material such as, for example, bone cement to become plastic, degrade, or deform in order to facilitate the removal or the repositioning of an affixed medical device within the bone cement. To this end, a novel composition of bone cement is disclosed, that in addition to its current formula, contains different weight by volume metal spheres, which respond to EMF and heat up the cement. These bone cement formulations may be composed of metal selections which match the implant material.

According to one aspect of the invention, an EMF emitting device comprises a wire system, a wire, and connector which is connected to a cooling and power system. This wire system may be designed to function with high current, high voltage, at low to very low radio frequency, to generate a strong EMF, and to generate custom fields. The wire system may be provided in a variety of lengths and diameters for different power levels and different application times. The wire system may be provided with insulation and may have EMF shielding along portions of the wire. In some embodiments, the insulation may have high thermal conductivity. In some embodiments, the wire system may be designed to be applied to a preformed disposable or reusable apparatus to help make a consistent shape and spacing of the working portion of the wire system. This apparatus may be patient-specific or have versions that are to be used in a patient-specific manner. This apparatus would be considered part of the wire system. In some embodiments, the EMF emitting device is designed to be coupled with a cooling system. In one such embodiment, the EMF emitting device comprises a wire system formed around a tube having high thermal conductivity designed to allow for the passage of a coolant through the system.

The wire system may be utilized in certain embodiments of treatment to make fields that vary in strength, by adjusting the distance or proximity of the wire to the point of treatment. In some embodiments this may be used to increase the heating speed in some areas, while in other areas, it may be used to lessen the speed of heating. In some embodiments, through superposition, the wire system may be utilized to focus the EMF to a target location.

In certain embodiments, the wire system further comprises one or more bendable elements that are capable of retaining a deformed shape. The one or more bendable elements may be positioned within the wire system, along the surface of the wire system or, in embodiments with exterior insulation, between the wire system and the insulation. The wire may change in plasticity as a result of a temperature change, thus rendering it more or less bendable. Thermally conductive, low-electrical resistance material may be positioned within or around the wire to facilitate cooling and to conduct electricity. In embodiments with a cooling system, thermally conductive, low-electrical resistance material may be wrapped around the tubing to facilitate cooling. Alternatively, in one embodiment, the wire system may be connected to the cooling system, power system, and a conductive pipe which is shaped variety of different shapes. Two or more such wire systems may be utilized to fully surround a portion of the body.

The wire system may be affixed to one or more quick connect/rapid disconnect fittings. The one or more quick connect/rapid disconnects may consist of male or female connectors, adaptors, or combinations thereof. In certain embodiments, the one or more quick connect/rapid disconnects may comprise a housing, one or more locking systems, one or more ball bearings, one or more springs, and one or more electrical contacts. The one or more quick connect/rapid disconnects are capable of connecting the wire system to a power system and, in embodiments employing a cooling system, the one or more quick connect/rapid disconnects are also capable of connecting the cooling wire system to a coolant source.

The housing of the quick connect/rapid disconnect may be made of a polymer or metal depending on the application and preferably has high strength, high hardness, high melting temperature, and a low thermal coefficient of expansion. The one or more ball bearings and springs of the quick connect/rapid disconnect may be made of metal. In embodiments employing a cooling system, the male and female connectors can contribute to the cooling of the electrical contacts by keeping the cooling tube in contact with coolant source and the wire.

The cooling system may be compromised of a liquid or gas cooling system. The liquid or gas cooling system may be regulated by a pump or by pressure, which is regulated by a control system in the power system module. The cooling system may be used to cool the wire system, making the wire system rigid, or to heat the wire system, rendering the wire system flexible. The cooling system may be connected to warm liquids or cold liquids.

The power system may comprise a wired power source, such as a cord attached to a power source, or a cordless battery power source. The power system may be characterized by a low/very low radio frequency generator/oscillator, variable voltage transformer, a rectifier, a LCD, physical switches, inverters, capacitor tanks, and a phase locked loop control system used to adjust the frequency of the oscillator to resonant frequency or detune the wire system to control power transfer, a phase shift control H-bridge, a liquid cooling system for the power system, one or more micro-controllers for the liquid cooling system for the power system, for the cooling system, and a user interface which may be digital LCD or physical switches. All control systems may be integrated into one micro-controller which interacts with all systems of the power system. All control systems may utilize other programmable logic devices or modules instead of a micro-controller. The power system may contain a variable voltage transformer which may be controlled by a micro-controller. The power system may be designed to directly integrate with the wire system via a female connector, which may be designed to allow fluid to flow into the wire system. The power system may be designed to utilize a variety of different power transfer limiting techniques other than limiting the voltage supplied, such as detuning the oscillator/inverter away from resonant frequency, modifying the PLL circuit to not resonant frequency lock, etc. The power transfer system may utilize a variety of sensors to monitor the load, resonant frequency, current, temperature, voltage, or other aspects of the system to provide safety measures, feedback loops, or other control feedback over the system. The system may be designed to control the cooling system for the wire system. The system is designed to emit variable strengths of EMF.

According to another aspect of the invention, an EMF emitting device is used to generate heat on the surface of a metallic implant in order to heat the material into which the implant is embedded to cause a change in plasticity, deformation, or degradation of the material in connection with the repositioning, insertion, or removal of the metallic implant from the body. According to another aspect of the invention, heat is applied to a patient to weaken a biological agent making it more susceptible to antibiotics and/or the patient's immune system. This can be accomplished by an EMF emitting device used to generate heat on the surface of a metal object for the purpose of heating surrounding tissues, biofilms, or planktonic bacteria. Heat may also be administered to the location of interest by other external devices and other methodologies.

### BRIEF DESCRIPTION OF THE DRAWINGS

FIGS. 1A-C show different embodiments of the wire system according to the present invention. FIG. 1A shows the cross-section of one embodiment of the wire system containing a high thermal conductivity cooling layer, a conductive layer, and an electrically isolating layer. FIG. 1B shows the cross-section of an embodiment of the wire system that includes bendable elements comprising a semirigid material. FIG. 1C shows the cross-section of an embodiment of the wire system that includes an electromagnetic shielding layer. One or more of these embodiments may be found within one wire system, and all of the layers may combine materials with a different layer.

FIGS. 2A-C show the cross-section of one embodiment of a quick connect/rapid disconnect component of the wire system. FIG. 2A shows a male connector inserted into a female connector. FIG. 2B shows just a male connector. FIG. 2C shows just a female connector.

FIGS. 3A-F show some exemplary orientations of EMFs that can be generated by the wire system. The darker shaded regions are EMFs generated by wires which are superimposed upon one another, and the lighter regions are EMFs generated by wires which are not superimposed upon one another.

FIG. 4 shows an exemplary wire system.

FIGS. 5A-G show data from studies of MRSA.

FIGS. 6A-D show data from studies of bone cement.

### DETAILED DESCRIPTION OF PREFERRED EMBODIMENTS

According to some embodiments, the EMF emitting device comprises a flexible, semi-rigid, electrically conductive wire system, a power system, and one or more quick connect/rapid disconnects. In some embodiments, the EMF emitting device further comprises a cooling system and/or partial EMF shielding.

Flexibility and semi-rigidity of the wire system are important features of the EMF emitting device as the created EMF must maintain a consistent shape in order to provide consistent heating over the generated EMF profile. The wire system must be flexible enough so that it may be formed into the desired shape to create a suitable EMF for the body part and application to which it will be applied, but sufficiently rigid so that the desired shape is maintained during the application of EMF. This may be accomplished by using materials that are temperature-sensitive and capable of retaining their shape when cooled. This may also be accomplished by adding one or more bendable elements to the wire. In certain embodiments in which a smaller diameter wire is used, tape, wrap, or a coating may be applied to the wire to assist in maintaining its shape. In other embodiments in which a larger diameter wire is used, the wire may be sufficient on its own to retain its desired shape. Still further in other embodiments, a disposable or reusable aid is used to orient the wire around the implant.

According to an aspect of the invention, the one or more quick connect/rapid disconnects provide the EMF emitting device with the ability of interchangeability of wire systems and to allow for the wire system to be disconnected from the power system, and the cooling system (if used), and configured or reconfigured around body parts with ease and speed. In addition, the one or more quick connects/rapid disconnects allow a wire system to be exchanged for another wire system for different applications. The quick connect/rapid disconnects may utilize a variety of rapid locking systems, such as a ball locking system, to quickly attach and rapidly disconnect a wire system. Other quick connect/rapid disconnects may utilize one or more of a variety of locking systems such as threaded locking systems, clamping locking systems, threaded and non-latching systems, bayonet latching systems, snap latching systems, non-latching systems, dry break disconnects, or other similar rapid attaching and disconnecting locking mechanisms. These quick connect/rapid disconnects may utilize electrical contacts in connecting the wire system to the power system. In embodiments which employ a cooling system, these quick connect/rapid disconnects may utilize a coupler or threading mechanism to connect the cooling system with a coolant source.

In certain embodiments, the EMF emitting device may further comprise a cooling system. In such embodiments, it may be desirable to cool the wire in high power applications in order to prevent the wire from melting or to dissipate heat so as to not burn the patient or medical personnel who handle the wire system. High power is useful in generating a strong EMF in treatment applications involving larger body parts such as, for example, a leg or hip. Strong EMFs may be necessary for creating certain heat curves, which may be more useful in some applications while in others, it may be less useful. In lower power or shorter time applications, a cooling system may not be necessary. In some embodiments, the cooling system may be configured in such a manner that, when activated, it reduces the flexibility of the wire.

In certain embodiments, partial shielding of the wire is provided. While the working portion of the wire cannot be shielded, some wires may have partial shielding in other portions of the wire so that the EMF is not applied to structures which are not intended to be heated. Partial shielding may generate more heat than unshielded wires as the EMF will also create current in the shielded material causing resistance heating.

According to one aspect of the invention, a set of one or more easily attachable and detachable wires are described which terminate with a compatible quick connect/rapid disconnect connector. The wires are designed to handle low to high power with combinations of about 1-10,000 volts and 1-10,000 amps resulting in different power limits for different wires and different applications. The assorted detachable wires may be from about 0.1 meter to about 100 meters in length and a diameter from about 1 mm-15 cm. The assorted wires are designed to handle both AC and DC power. These variables will affect the power rating, the operating frequencies, the application, and the composition of each wire. This combined system is intended to generate a 10 mT-5 T field at the focal point of the super imposed fields depending on the settings of the power system, the metal intended to be heated, and the orientation of the wire. The detachable wires are designed to be easily connected and disconnected from the power system, control system, and cooling system, and to be easily configured around a body part of a patient or object for application of an electromagnetic field. The EMF generated by the system can be customized for the individual use case. This is especially important because bone cement is not always used in thickness of the ideal application, because of deformities in the bone in which the implant is going into. This is especially true in revisions. While the wires may take a variety of shaped forms, certain patterns are preferred for some use cases which have relative focal points for the EMF. Such configurations may include a cylindrical coil, a hemisphere, a zig-zag configuration, or a 2- or 3-dimensional spiral configuration. In certain embodiments, the wire is designed to be very flexible, while in other embodiments the wire is designed to be semi-rigid or follow an inflatable guide, a disposable/reusable guide or similar aid capable of holding the wire's form in which it is shaped. This disposable guide in some embodiments may be utilized as a means to rapidly deploy a complex AI generated wire system geometry which generates a complex EMF to cause a strong field superposition in some areas while a weaker one in others, thus causing intentional uneven heating on the surface of the metal, which can be used to not only heat up an implant to a certain value over a certain period of time, but also heat the attached bone cement to the same values simultaneously.

The AI or the technician or surgeon will need data points on how to construct or use preexisting guides for the treatment. Some of the useful preoperative workup for PJI includes the use of differential imaging studies as well as organism identification either with culture and sensitivity or 16s DNA deep sequencing and a variety of other labs. The preop imagery includes plain radiographs that often show radiolucency around a prosthesis or radiodensities that can be indicative of infection or aseptic loosening such as wear and debris responses. Radiographs are the first and the least expensive imaging option for helping develop a localization of responses from the host to the infected area involving prosthetic joint replacement. Due to this it is often used. In addition to this, bone cement is also displayed on radiographs, which can establish thickness in some local regions. Tri-phase bone scan is very helpful for localization of the infection and bone cement as well. The early vascular phase depicts the reactive soft tissue involvement around the joint with the delayed phase showing more of the bony involved areas using technetium 99 as the isotope. Ceretec or technetium 99 labelled white blood cell (WBC) scans, which take 24 hours to complete, can depict well bony and soft tissue involvement when the infection has pus or abscesses. It is less useful in scenarios where the infection is related to pure biofilms, however this can change treatment specifications, as planktonic bacteria is present. SPECT scanning gives better localization with either of these two nuclear medical scans. Computerized Axial Tomography (CT) gives a much better resolution of involved areas of periprosthetic Joint Infection (PJI) but has limitation around the metallic device secondary to electron scatter effect. With subtraction technologies employed CT scans can show the extent of soft tissue involvement as well as bony involvement around stem but is limited around bulky prosthesis like distal femoral replacements and tibial baseplate. A combination of these four studies is recommended as part of the workup for PJI. MRI has little to offer because of image degradation. Other useful data for generating customized EMFs, disposable guides, and wire orientations may include the type of implant, the metal type, previous antibiotic treatments, and patient-related measurements such as thickness of body part. All of these preoperative workups and information can provide valuable data points which can influence the treatment settings of a patient, the geometry of the wire system, and the use/construction of the disposable guides.

The data set may be utilized by an AI or technician to generate patient specific information, such treatment field strength, spatial orientation of rapid deployment guides, time of treatment, and custom EMF. With the use of complex imaging, it may be possible to locate exactly where the biofilm of interest is on the implant and bone cement. With the use of complex imaging, it may be helpful in treatment protocol development to recognize exact thicknesses of bone cement, as biofilms may grow on bone cement and may require treatment. In addition, surgeons often use more or less bone cement in areas where deformities are present in the bone of implantation or from previous revision, this may add to the complexity of the personalized treatment and, if ignored, may render treatment plans/settings ineffective if not accounted for.

The present invention will allow for precise treatment, to be as non-invasive as possible. Alternatively, the surgeon or trained technician may orient the wire system using the knowledge from the imagery to account for the abnormalities or to focus the field on the site of infection, using the partially rigid nature or preformed materials of the wire system. This may be done with one wire system or multiple wire systems. The disposable guide may be used to hold the wires in position. Wires intended to be used with high power or for long treatments may be designed to be integrated with a cooling system. Wires intended to be used with lower power settings or for shorter treatment times may not need to be integrated with a cooling system. The wire system of the present invention allows for wires of different dimensions and cooling features to be used with the same power, cooling, and control systems.

In certain embodiments, the wire may have electrical insulation on the outside and some may have high thermal conductivity, allowing the wire to be used as a cooling element if the cooling system is activated and the power system is deactivated, and as a heating element when the power system is activated and cooling system is deactivated. This allows for the EMF emitting device to be used as a heating or cooling instrument depending on the application. In certain embodiments, the wire may have insulation with low thermal conductivity to block heat transfer so that heat is kept away from a patient or other objects. There is no electromagnetic shielding on the working portion of the wire.

In certain embodiments, the wire may have EMF shielding on a portion not designated as the working portion of the wire so as to not emit an EMF where it is not desired such as, for example, an operating table. The working portion of the flexible wire is the portion of the wire designed to emit an EMF which may be marked on the outside of the wire or by a specific connector. Such markings help the medical provider know where the working portion of the wire is located.

According to one aspect of the invention and disclosure, the bone cement may be impregnated with metal particles or spheres of a size between about 1 mm to about 1 nm, which respond effectively to an EMF causing the bone cement to heat up when exposed to a current inducing EMF. In certain embodiments of the bone cement, the metal is designed to match the type of metal used to manufacture the implant in which it affixes. The embodiments of the bone cement formula change in formulation by weight. Quantities of metal particles will be from less than about 1% by weight of the formula up to about 50%. PMMA, or other like materials which are commonly found in bone cement will make up the rest of the formulation. The purpose of matching the materials, is to cause similar heating curves that would be seen on the implant's surface, which would allow the whole prosthetic and bone cement to be heated without having to alter the field strength where bone cement is located for even treatment of microbes in the area or biofilm, as well as explanation in the case of prosthetic failure. This will be useful for all future implantations that are designed to use this model. It's also quite useful in that bone will be easily removable if explanation or revision is needed.

According to one aspect of the invention, the wire system connects to a power system which is connected to a wired or a battery power source. The power system may have a micro controller or programmable logic device/module which interacts with a single phase, 2-phase, 3-phase, etc. variable voltage transformer and the LCD to control the incoming voltage to the power system. The user interface may consist of an LCD or a series of switches and dials. This system is designed to convert AC current, in the case of a wired approach, to DC using a rectifier(s). The signal may be smoothed by inductors and a capacitor tank for a clean signal. The use of a variable voltage transformer is a very important feature for an AC lined system, as it allows for the voltage to be adjusted on the front end of the system. This will directly affect the current flowing through the wire system, which directly affects the strength of the electromagnetic field. In the case of DC, this voltage may be regulated by connecting different batteries via a series of switches. If the device is powered by batteries the system may be controlled by a microcontroller, a programmable logic device/module, and/or a switch(s), to add batteries to the system to increase the available voltage. In both cases increasing the voltage is very important, as the current that is in the wire system affects the EMF strength. At a very fundamental level, by modulating the current flowing through the wires, it can increase or decrease the field strength and the distance it may travel. Based off the interaction between these fields and the orientation of the wire, it can change the resonant frequency significantly. An orientation that is not relevant due to wire spacing with a low strength EMF, may be very relevant if the current is increased because the field strength increase will allow for a significant interaction to occur between nearby wires. As such, the resonant frequency of the wire system can thus be changed by modulating the current over the system. This may also be relevant in achieving the necessary field strength to heat an implant or bone cement over a distance, even if fringe fields are used in the heating process. In the case of both the wired system and the battery system, the clean DC signal is then delivered to oscillators/inverter circuit, which may be in an H-bridge, which may have IGBT(s), MOSFET(s), or a similar oscillator component(s) in it. An H-bridge may be utilized with the oscillator components which may be used for frequency tuning of the wire system and capacitor tank to resonant frequency or detuning it to reduce current and field strength. These components and circuits may be connected to a set of circuits and a micro-controller or programmable logic device/module which are capable of finding the resonant frequency wire system and capacitor tank and use a phase locked loop (PLL) which may be used to modulate optimal power transfer to the metal object or to adapt to a new configuration of the wire system. This is a very important feature of the power system, as it allows the wire to be used in many configurations and optimizes power transfer to metal objects. The same microcontroller or control module or an additional one monitors the temperature of the cooling system lines, incoming and return lines, to make sure the system is appropriately cooled. This system is also capable of having the cooling system to the wire system turned off or turned on separately from the cooling system for the power system.

$${q}_{x} = - k A \frac{( {T}_{w} - {T}_{s} )}{x}$$

The above equation is for heat transfer at a wall. When looking at this system, the transfer of heat from the implant to the surrounding materials is the interest of this application. In this case T_{w} would be considered the temperature of the implant and Ts would be the temperature of the surrounding material. One of the best ways to increase q is to use induction heating to rapidly raise T_{w}. A represents the area, x represents the distance the heat will travel, and k is a constant related to the materials heat transfer properties known as the thermal conductivity constant or coefficient of heat transfer which is specific for each material. Significant power transfer can happen using induction heating, much faster than most types of heating methodologies. Due to this, q can be higher than in most other applications at a much faster rate. Due to using a low/very low radio frequency, the temperature of the implant is distributed beyond just the surface, which allows for even heating of the implant and tapered cooling of the implant. In addition, it allows for more controlled heating. This allows for a rapid heating curve of the surrounding environment which is capable of inducing shock to biofilms and planktonic microbes. This equation is very relevant when heating through bone cement as well.

Each implant material will have a different heating curve and some materials such as titanium will heat much slower and thus the field generated must be variable in nature. The power system is modeled after an induction furnace, which utilizes frequencies of operation being 1 KHz-100 KHz, very low/low radio frequency, so as to generate a magnetic field that is known to heat the top 1-5+ mms of the surface of a metal depending on the type of metal and the field strength. This allows for even heating even on contoured pieces and an even rapid heating profile. This also allows for high strength fields to be utilized effectively, necessary for large diameter configurations of the wire system, such as around large leg with excessive fatty tissue, where location of targeted heating, may be on the fringe of the field.

Lower power, higher frequency systems were considered for the power system but ultimately were chosen to not be used as they are specialized in heating smaller objects at a skin level, fractions of a millimeter, in close proximity to the coils or wires. These systems are not ideal for heating implants and bone cement for several reasons. First, when implants are manufactured, they are often machined. Machining adds mechanical stresses to the metal, known as stress risers, which when exposed to an induction field are known to heat up faster than the metal which do not have the stress risers on the same work piece. This causes uneven heating of the implant and heat spikes to occur whenever they are exposed to an EMF, but especially so when exposed to higher frequencies, which utilize skin heating. To avoid significant stress riser heating due to defects in implant due to machining, it was chosen to utilize lower frequency waves which penetrate further into the metal, which when used can provide more uniform heating. In the case of human flesh high frequency EMF used for this application may cause isolated tissue damage. In the case of bone cement this may cause it to burn. In addition, high radio frequency systems typically have a skin effect which does not cause even surface heating over contours. Lower frequency fields, penetrate to a depth of the metal and are not superficial. To this end, very low/low radio frequencies from 1 kHz-100 kHz were chosen for microbial treatments, implant removal or replacement, bone cement removal, or other metal object treatment.

In one embodiment, the power system does not have preprogrammed settings, as applications of heating implants will be different, and the required field strength and power transfer will need to vary. In one embodiment, a variable voltage transformer or batteries are used as the means of power control which can control the overall strength of the EMF emitted. By controlling this parameter, one is able to effectively control some of the power transfer to the metal object as well as the distance the field travels. There may be predetermined range of settings for the voltage and frequency which may be chosen to correlate with the type of metal of the implant, the manufacturer, the diameter of the body part or distance to treatment site, and the orientation of the wire. An AI may also generate these settings based off the given variables. The technician/surgeon will need to adjust these settings in response to prework imaging, to create a patient specific treatment. In the case of an AI, the data related to the pre-work-up may be used to generate voltage and frequency settings, as well as other aspects of the treatment. The oscillator may also have a fixed starting frequency for a particular type of metal. The system is designed such that it integrates and adapts to the wire system's geometry. The wire geometry may be changed because bone cement is not even across the implant. While the instructions for applying bone cement recommend placing a millimeter or two of bone cement on the implant, during surgery, surgeons often have to place more cement in certain areas due to defects.

The cooling system in some embodiments utilizes a liquid cooling system with a variable speed pump, temperature sensors which monitor the fluid temperature of outgoing and incoming lines, a reservoir, a radiator, and a fan. This cooling system may be connected to the power system and may interact with a micro-controller. In other embodiments, the system may utilize a gas cooling system which may be controlled by pressure release using an automated regulator valve controlled by a micro-controller, programmable logic devices, or modules which communicate with the power system or is located in the power system. The cooling system may also be a combination of liquid and gas systems, which are cooling components of the power system and the wire system.

When the above-described systems are connected and utilize a variable low radio frequency, variable high-power EMF can be generated and applied in a variety of configurations and strengths to a metal object(s) in a controlled manner, such as an implanted device, plate, or screw, which can be heated through the body, clothing, bandages, and other non-metal materials at a distance. In one embodiment, the EMF emitting device emits an EMF which heats the surface of an implanted device such that it, in turn, heats adjacent temperature-sensitive material, such as bone cement, to facilitate repositioning of the device within or removal of the device from the cement. The emitted EMF causes an electrical current to form on the metal surface which, due to electrical resistance, creates localized heat which can soften or weaken the bone cement to allow the surgeon or technician to reposition, insert, or extract the metal implant more easily. Alternatively in another embodiment, the bone cement itself, is impregnated with metal particles which allow for heating, repositioning, removal of implant, or removal of bone cement.

The wire system described above is useful in surgical revisions because it allows the surgeon to rapidly configure the wire around the body part of the patient in the operating room without having to significantly maneuver the patient. In addition, the wire system can accommodate different sizes of bodies in terms of both being able to switch wires for a shorter or longer length, and to be able to wrap around thicker or thinner body parts or create more turns around the patient. It also allows for a custom focal point to be made, which can keep some areas cool, which may be in contact with tissue, while other areas of the implant can be affected by the emitted EMF heating up only a portion of the implant, which may have bone cement over it. It also allows for EMF field to be intensified at the fringe of the EMF by changing the diameter or the spacing of the turns/pattern.

When the above-described wire system is attached to the AC power generating system, an EMF field can be emitted causing heating of metal objects such as metal implants, screws, plates, and the like. The localized heating of the surface of these objects can also cause infectious agents to become more sensitive to antimicrobial agents, the immune system, and/or the environment. Such a device is particularly useful because it allows an infection, such as a biofilm growing on an orthopedic implant, to be treated non-invasively. In addition, as in the above method, it can accommodate different size bodies in the same manner as well as be easily deployed.

In another embodiment, a treatment method utilizing an EMF emitting device can disrupt the growth of algae and even destroy it. At wastewater treatment facilities, high intensity UV light is emitted through a transparent portion of pipe or other architecture, usually glass, in order to kill viral particles and/or other microorganisms. This light becomes less effective as algae forms. A wire as described above can be deployed rapidly and easily without disconnecting any of the inline pipe to heat and kill the algae. If a strong enough field is present, even glass is capable of melting from these EMFs, and heating such a material is quite possible. Alternatively, an EMF emitting device may be used to heat inline water (in a metal or glass pipe) and kill pathogens passing through, removing the need of UV light altogether. In addition, this would allow for some pharmaceutical compounds in wastewater to be broken down such as testosterone and estradiol, which has been linked to a variety of health issues for animals and humans. This heated water can be cooled (or condensed from steam) as the temperature downline is cooler. Such heat treatments and cleaning methodologies can easily be performed using this described treatment plan and embodiment of the invention. The rapid disconnects/quick connects allow for this system to be deployed on existing infrastructure without needing to open the pipe to get the wire around the pipe, thus allowing for multiple areas of piping to easily be serviced.

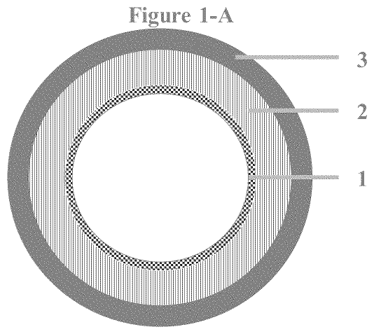

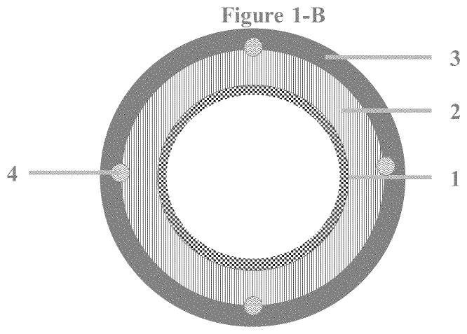

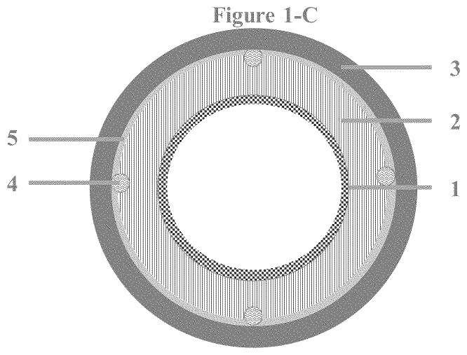

Another embodiment of the invention is the impregnation of bone cement with micro particles of metals. In certain embodiments, the metals are designed to match the alloys magnetic permeability so as to allow even heating of the bone cement at the same magnetic field strength and can potentially reduce the complexity of the treatment processes. Such metal particles may be titanium, cobalt, chromium, steel, zirconium, and other common implantable metals and their alloys. Such an embodiment also allows for the bone cement to be removed from the body, as it is now capable of being heated independent of the implantable device. This ability to heat also aides in its ability to weaken biofilms and planktonic bacteria in the surrounding tissue such that they become susceptible to antibiotics. FIGS. 1A-C shows cross-sections of the wires or portions of the wires in the wire system. These wires can consist of any number of these cross-sections within the same wire. The thicknesses of all cross-sectional components may vary based on the needs of specific applications, such as higher or lower current requirements, longer wire length, the number of coils, the diameter of coils, spacing of coils, longer or shorter application time, heat conductivity, etc. All cross-sectional components may change in diameter due to patient size and/or field strength needed. All parameters can be adjusted to fit the size of the patient and the EMF needed. It should be noted that some of the materials can be moved within the cross-section of the wire and may not be placed exactly as pictured. Some materials may be combined. Lastly, it should be noted that in some embodiments, some materials may not be present based on application needs.

In FIGS. 1A-C, the cooling tube 1 is designed to drain heat from the wire system and/or provide cooling to the surrounding environment of the wire. The cooling system may also be designed to drain heat in order to prevent the wire system from heating up too much and burning the patient or melting materials near the wire system for the given application. Cooling tube 1 may be designed to have a variety of different cooling materials to flow through it, such as ice water, refrigerant, liquid/gas CO₂, liquid/gas nitrogen, or another coolant suitable for the application. Cooling tube 1 made be made from any suitable material that allows it to be flexible, to undergo repeated thermocycles and cyclic bending, and to have high thermal conductivity. Exemplary materials for cooling tube 1 include polytetrafluoroethylene (PTFE), ethylene tetrafluoroethylene (ETFE), fluorinated ethylene propylene (FEP), perfluoroalkoxy alkanes (PFA), polyphenylene sulfide (PPS), liquid crystal polymers (LCP), polyphthalamide (PPA), polycarbonate (PC), nylon 66, polypropylene (PP), polyethylene (PE), thermoplastic elastomers (TPEs), or other materials that provide the foregoing properties. In applications which use high power, high thermal conductivity is particularly useful. In addition to previously recited materials, cooling tube 1 may be made of a thermally conductive metal which also has low magnetic permeability but is more rigid than polymers. Such metals may include copper or aluminum which have high thermal conductivity (100-500 W/m·K). In addition, some thermally conductive materials common in thermal pastes and adhesives may be used to increase thermal conductivity and increase conduction between layers.

Conductive layer 2 preferably has a low resistivity, sufficient thickness to conduct the current for the application, good thermal conductivity, and is flexible. Conductive layer 2 is designed to carry sufficient electrical current to produce an EMF, dissipate heat, and retain its flexibility for the specific application. Conductive layer 2 may be made of silver, silver alloys, copper, copper alloys, gold, or another material with similar material properties. Conductive layer 2 may be solid or comprise a plurality of individual smaller wires.

Insulation layer 3 is designed to make the system electrically isolated. Insulation layer 3 preferably has low electrical conductivity and is flexible or semi-rigid to allow the user to bend or conform the wire to a certain shape and for that shape to be retained. For some applications, it is desirable for the insulation layer 3 to have high thermal conductivity. In other applications, an uninsulated wire may be used to heat its surroundings through resistance as well as cool the surroundings as the cooling system is present. Insulation layer 3 may be made of any suitable material including rubber, silicone, perfluoroalkoxy alkanes (PFA), polytetrafluoroethylene (PTFE), mica, fiberglass, combinations thereof, or other materials having similar properties.

In FIGS. 1B and 1C, support members 4 are made of a semi-rigid material. Support members 4 allow the user the ability to conform the wire to a shape but offer resistance to changing from initial shape to a different shape. Support members 4 may be made of separate individual rods (as shown) or alternatively may comprise a continuous layer. Support members 4 can be made of glass rods, fiberglass, glass polymer composite, combinations thereof, or other materials with similar properties.

In FIG. 1C, electromagnetic shielding layer 5 is designed to minimize the interaction of the generated EMF with nearby objects. Electromagnetic shielding layer 5 is preferably flexible, exhibits high electrical and thermal conductivity, and is capable of effective EMF shielding. Electromagnetic shielding layer 5 may be a continuous solid layer or a mesh. Electromagnetic shielding layer 5 may comprise copper or a coppery alloy, a silicone metal composite, aluminum or an aluminum alloy, silver or a silver alloy, nickel-graphite, combinations thereof, or other materials with similar properties.

All of the aforesaid components of the wire system may use some form of a bonding material to connect them to other components at any point in the wire system. The different layers of the wire system may be combined or positionally switched with other layers. The above list of materials is provided as examples and is not intended to be exhaustive as many other materials have the desired properties and can be substituted.

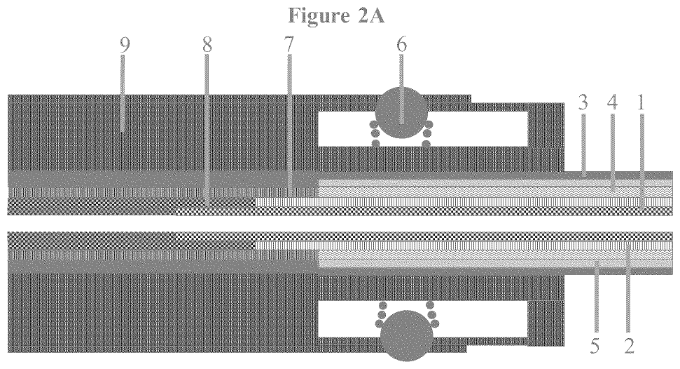

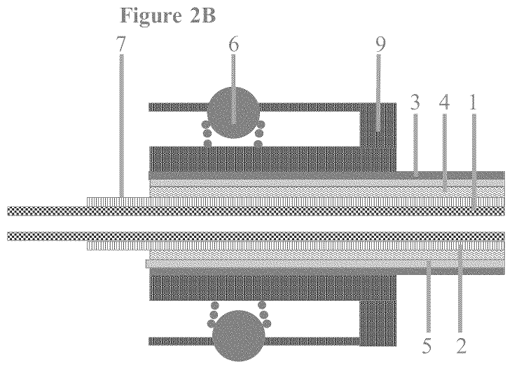

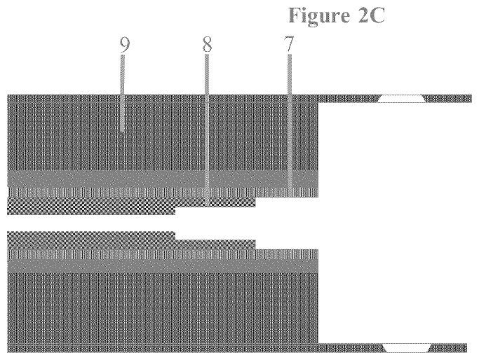

FIGS. 2A-C show cross-sections of the quick connect/rapid disconnect connectors and the terminal portions of the wire system within the connectors and their connection to power and cooling systems. The cooling system and power system terminate in the female connector while the wire system terminates in the male connector. The female connector may integrate with the male connector at the coolant interconnect, the electrical contacts, and the housing. In certain embodiments, the connector may have one or more additional and/or a different rapid locking systems other than a ball bearing locking system.

Ball bearing locking system 6 comprises a ball bearing, a partially compressed spring, and a hole which allows the ball bearing to lock into place. The materials for the ball bearing and the spring may be low-alloy steel, cold formed steel, oil tempered, bainitic hardened steel, stainless steel, copper and titanium alloys, combinations thereof, or other materials exhibiting similar properties or similar performance.

Electrical contacts 7 may be brushed or polished smooth to enhance the electrical connection. The electrical contacts 7 are provided on both the male and the female connectors and are in electrical connection with a power system and the wire system. The electrical contacts 7 may be made of silver, copper, gold, platinum, palladium, tungsten, nickel, graphite, molybdenum, an alloy combination thereof, or of a similar material with similar material properties. The EMF emitting device may have a mechanism that applies pressure to the electrical contacts 7, such as a ball bearing spring locking mechanism as described above, to ensure an electrical connection is maintained.

Cooling interconnect 8 may be a coupler. This coupler may interact with the incoming wire of the wire system as shown or differently and may also be secured by a locking system or threading. Cooling interconnect 8 may be made from the same materials discussed for cooling tube 1.

Housing 9 may be made from a high strength, high hardness material having a high melting point, and low thermal coefficient of expansion. Housing 9 may be shaped as depicted in this embodiment or make take on other shapes. Housing 9 may contain other locking mechanisms instead of the portions which interact with ball bearing locking system 6. Housing 9 may have threading to thread with the male and female connectors. Portions of housing 9 may be capable of turning independently of other portions. Materials of housing 9 may be metals or polymers. Such materials may be stainless steels, aluminum alloys, resins, thermoplastics, combinations thereof, or a material with similar properties.

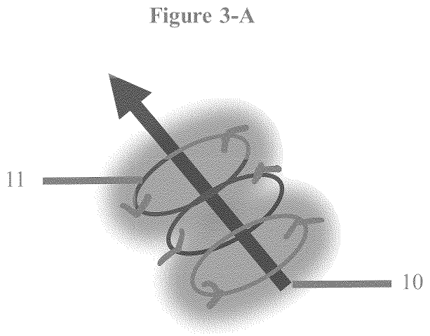

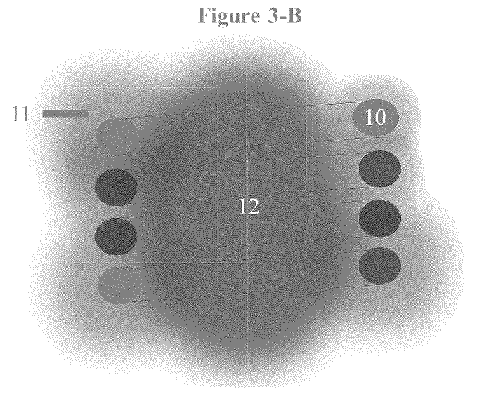

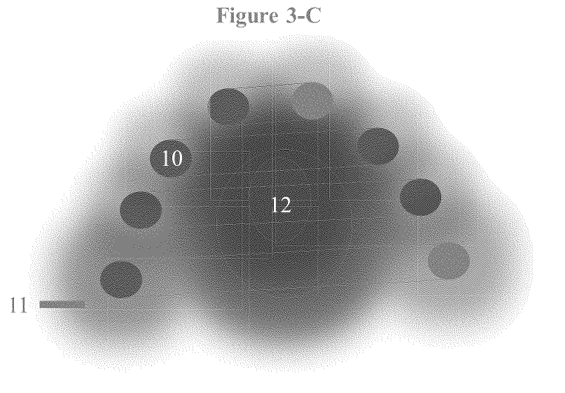

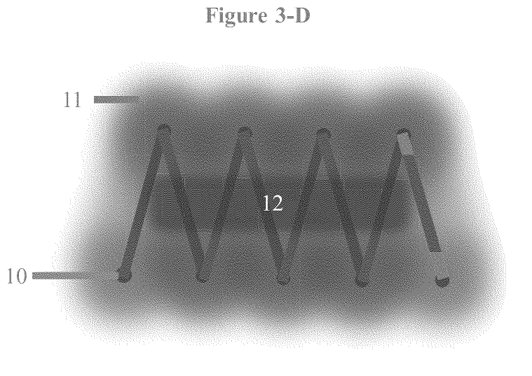

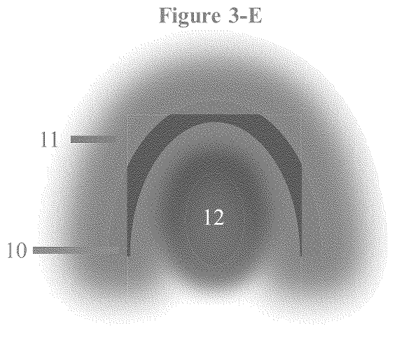

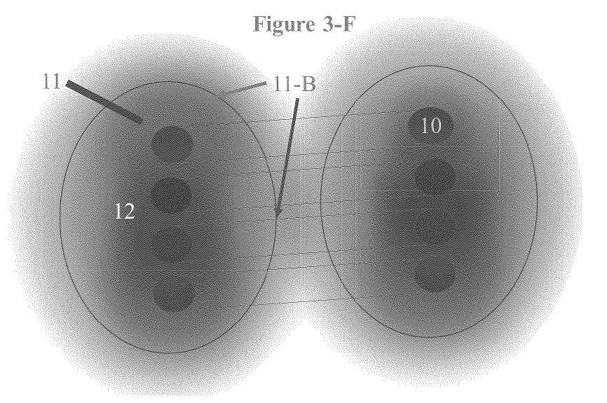

FIGS. 3A-E show some of the different ways an electromagnetic field can be deployed by a flexible or semi-rigid wire. FIG. 3A shows wire 10 emitting an EMF 11. FIG. 3B shows a cross-section of wire 10 in a helical coil configuration emitting an EMF 11. A resultant EMF 12 can be seen within the helical coil in the darker shaded area where the field would be more concentrated. Resultant EMF 12 is due to the overlapping of the EMF produced along the helical coil. FIG. 3C shows a cross-section of an alternative helical configuration which is suitable for use on a patient's hip. FIG. 3D shows a wire in a zig zag configuration which is suitable for use over a wound or portion of the body. FIG. 3E shows an end view of the wire configuration shown in FIG. 3D. FIG. 3F shows the same orientation as FIG. 3B, but the diameter is much greater and thus 11B, the fringe EMF, may be used in the induction heating process of implants, where the body mass near the treatment site does not allow for close proximity or placement of the wire system.

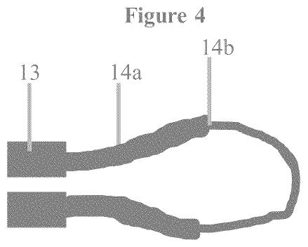

FIG. 4 shows an embodiment of the wire system comprising wire 14 and quick connect/rapid disconnects 13. In this embodiment, 14a contains electromagnetic shielding and thus is shown to have a greater diameter. At 14b in this embodiment, the diameter changes indicating that the wire has transitioned from being shielded to having no shielding. The unshielded portion of the wire is considered the working portion of the wire is to be manipulated to create the desired EMF. Alternatively, the change to 14b may be indicated by a connector, notch, or other marking at 14b.

### EXAMPLES

Example 1. Use of device to treat metal implants that have acquired biofilm or other microbial infections. This example also shows that heat treatments can render biological agents weakened and more susceptible to anti-microbials, their environment, and/or the immune system.

Approximately 3-5% of surgical implants such as artificial joints, screws, and plates become infected with bacterial and/or fungal contaminations. Often such infections are in the form of biofilm and are resistant to treatment with antimicrobial agents. These infections may become life-threatening and can require that the implant be removed, the site debrided, and a replacement implant installed. Such surgical operations to perform a replacement are time-consuming, technically difficult, costly, destructive of bone, and risky to the patient. A non-invasive method of treating infected implants is described.

The EMF emitting device of the present invention can deliver a focused EMF to metallic implants which causes localized surface heating of the implant. Because biofilms can adhere and grow on implants and other infectious material may be closely associated with the implant, they are exposed to the heated surface of the implant. While heating an infectious agent to temperatures that do not cause untoward damage to human tissue does not always kill the infectious agent, the EMF emitting device increases the sensitivity of biological/infectious agents to environmental factors such as antimicrobials or elements of the immune system. In this example, we present data showing that increasing the temperature of metallic objects causes Methicillin Resistant Staphylococcus Aureus (MRSA) biofilms to become more sensitive to levels of vancomycin to which it would otherwise not respond.

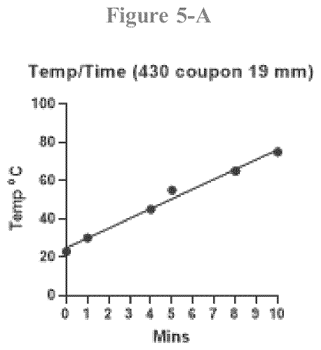

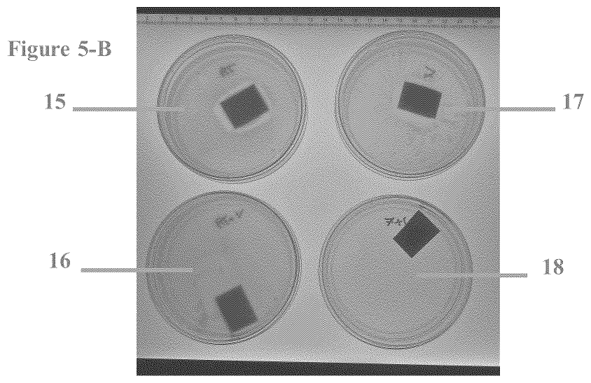

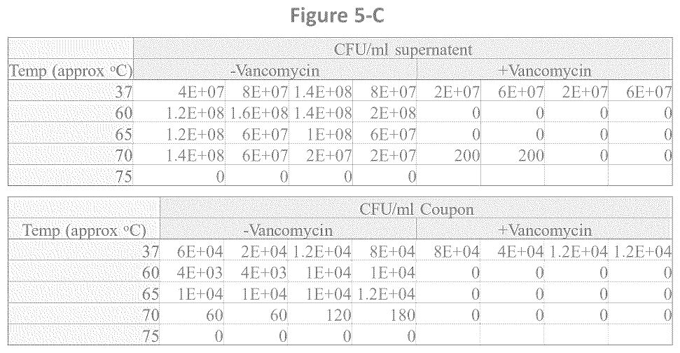

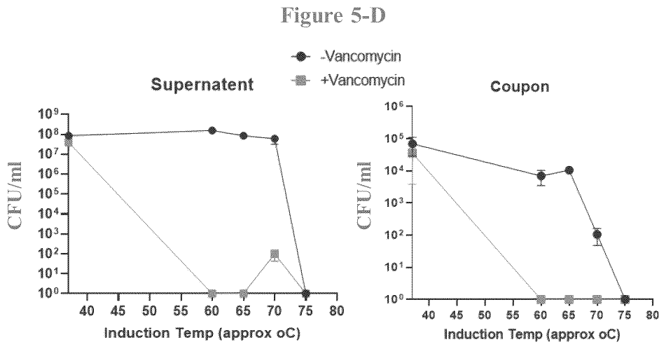

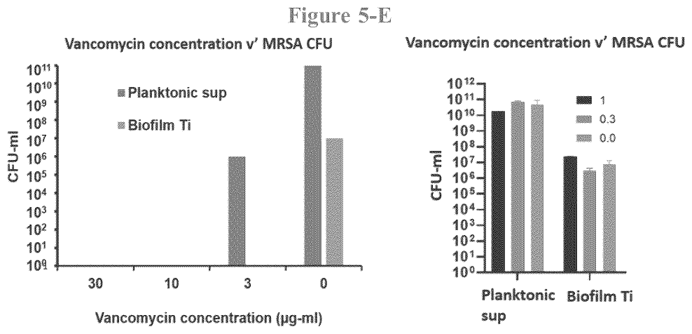

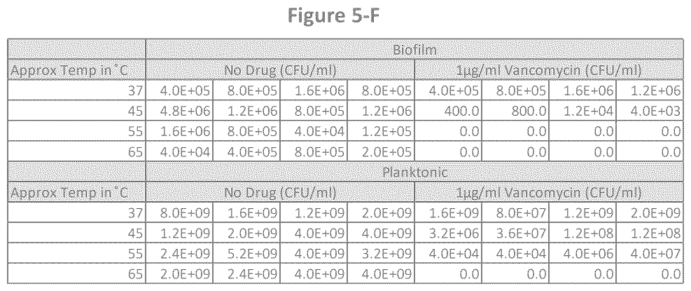

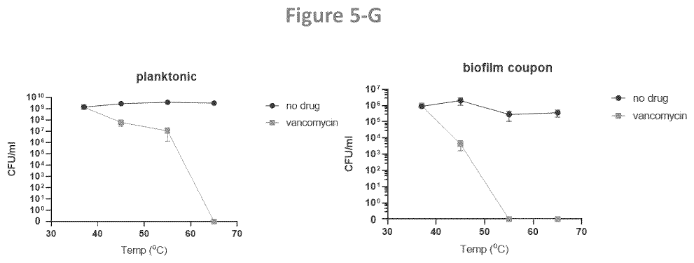

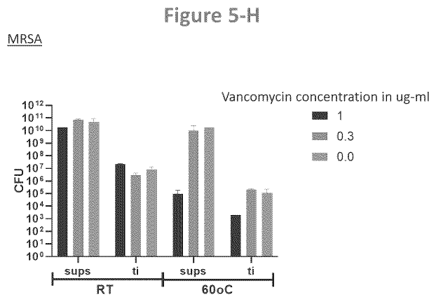

FIG. 5A shows a temperature-over-time graph performed using small metal bacterial growth surfaces, termed “coupons,” composed of 430 stainless steel. Each coupon measured 19 mm×25.4 mm×2 mm. The coupons were placed into sterile 50 mL tubes containing 40 mL of Tryptic Soy Broth (TSB) and subjected to heating with an EMF emitting device in accordance with the present invention. The EMF device was set to utilize 50-100 KHz and generated an EMF of 10.7 mT and a reasonable heating curve in the material. A glass thermometer with nonmetal filler was used to measure temperature over time. This experiment was used to determine the approximate time required to heat the coupon to a specified temperature.

A second study determined the vancomycin sensitivity of MRSA strain M2 that was used in these studies. Briefly, the bacteria were cultured overnight in tryptic soy broth (TSB) containing increasing concentrations of vancomycin. The preliminary study determined that a concentration of approximately 3 micrograms per milliliter (3 μg/ml) of vancomycin reduced the growth of the bacteria by approximately 50%. The revised Clinical Laboratory Standards Institute (CLSI) guidelines define that an S. aureus strain with a minimum inhibitory concentration (MIC) of ≤2 μg/ml as vancomycin-sensitive S. aureus.

A follow-up experiment was performed in quadruplicate to assess the effects of induction coil treatment on MRSA M2. Type 430-steel coupons were cultured with MRSA underneath TSB media for one week at 37° C. The media was changed on days 3 and 6. The coupons were removed from the culture container and washed 4 times using 25 mL of sterile phosphate buffered saline (PBS). The washed coupons were then transferred to 50 mL tubes containing 40 mL TSB media. Coupons were set aside as room temperature controls without heating, with and without 1 μg/ml vancomycin. Replicate coupons were each heated individually for 7, 8, and 9 minutes to approximate temperatures of 60° C., 65° C., and 70° C., respectively. In addition, a set of coupons were heated 10 minutes to a temperature of 75° C. Following heat treatment, the coupons were incubated in petri dishes containing TSB overnight at 37° C. with or without 1 microgram per mL vancomycin. After overnight incubation, the bacteria in the culture supernatant were enumerated directly by plating on agar plates using 10-fold dilutions. The coupons were washed four times with PBS. Bacteria were removed from the coupons with a plastic cell scraper and titered on agar plates using 10-fold dilutions. The data shown is representative over three similar experiments.

FIG. 5B shows a photograph of an example of the plates used to culture the coupons after induction coil treatment. Control coupon 15 was not heat-treated and was cultured without vancomycin. A large number of bacteria grew on coupon 15 and it can be seen as a murky/cloudy, deposit/colony on the plate. Coupon 16 was not heated but was cultured in 1 microgram/ml vancomycin. The appearance of the culture in coupon 16 closely resembles coupon 15 because the bacteria is capable of growing in 1 μg/ml vancomycin and is not inhibited. Coupon 17 was heated over a 7-minute period to approximately 60° C. and was cultured without vancomycin. The film of bacteria appears to be less dense than on coupon 15. Coupon 18 was heated over a 7-minute period to 60° C. and cultured overnight in 1 microgram/mL of vancomycin. The fluid around the coupon in 18 appears to be clear indicating little, if any, bacterial growth. Titers confirmed undetectable growth on coupon 18.

The bacterial growth was quantitated as described above. The concentration of colony-forming units (CFU) per mL is presented in the tables and graphs shown in FIGS. 5C and 5D. The upper table shows the CFU/mL of MRSA found in the supernatants of the quadruplicate samples. The upper graph shows the mean CFU/mL with error bars (standard error of the mean) of the data in the table. As can be seen, the CFU/mL remains relatively constant without vancomycin when the coupons are heated to 70° C. However, when vancomycin is included in the overnight growth media, the CFU/ml is strikingly reduced to or nearly to undetected concentrations. Similar results are shown in the lower table and graph. The amount of CFU/mL scraped from the coupons remains relatively constant without vancomycin to 65° C. However, when vancomycin is included in the overnight culture media, the number of CFU/mL is undetectable in this experiment. The data also show that heating the coupon to 75° C. is sufficient to kill the supernatant bacteria without antibiotic and that 70° C. reduces the CFU by approximately 100- to 1000-fold without antibiotic.

The foregoing results demonstrate that induction coil heating of biofilm-coated metal coupons renders the bacteria more susceptible to vancomycin treatment. It also shows that heat can be used to weaken biological agents and render them more susceptible to antibiotics and/or the immune system.

FIG. 5E shows representative data from experiments similar to those shown in FIGS. 5B-D. MRSA was cultured for 7 days on titanium plates at 37° C. as described. Plates were then washed and cultured overnight at 37° C. with no induction but in the presence of the indicated vancomycin concentrations. This experiment generated a baseline for MRSA M2 susceptibility to vancomycin in this system which agrees with the literature and current definitions of vancomycin-resistant MRSA.

FIGS. 5F-G show another experiment performed at lower temperatures. 430 steel coupons (19 mm×25.4 mm×2 mm) were cultured with MRSA under 1×TSB containing media for 1 week at 37° C. The media was changed every 3-4 days. Coupons were collected and washed four times in 25 ml PBS by gentle inversion and placed in a 50 ml tube which was filled with 20 ml of TSB. The coupons were then treated by subjecting them in an AC EMF for an amount of time which correlated with previous calibrated heating times and curve shown in FIG. 5A. In addition, temperature was verified using a handheld infrared thermometer. Vancomycin concentration was increased from 0 to 1 μg/ml in respective samples where its presence was indicated for the experiment. Following the treatment, the coupons were decanted with media into a petri dish which was topped off with an additional 20 ml of TSB. If vancomycin was indicated for the particular sample, the vancomycin concentration was maintained when the addition of the 20 ml of the TSB was made. These samples were then incubated overnight. Supernatants were collected and CFU counted directly by plating the TSA. Biological replicates, two or more coupons per condition were sampled, then titrations were performed per coupon and supernatant of each sample. The data generated from the replicates were used to verify the CFU for the biofilm and the supernatant. Coupons were further washed four times and resuspended in 1×PBS and then scraped with a cell scraper. The solution was then serially diluted, and microtiters performed for the CFU on TSA plates using 5 μl per dilution for 10-fold dilutions starting at zero. FIG. 5G reports the CFU/mL bacterial titers of planktonic (left) and biofilm (right) bacteria incubated with or without 1 μg/ml vancomycin after EMF treatment raised the temperature of the coupon from 40 to 65° C. The standard error can be seen in FIG. 5G. As can be seen, the increased temperature had little effect on the CFU/mL when the bacteria were cultured without vancomycin. However, an increase in temperature from 55 to 65° C. resulted in a reduction of CFU/ml of more than 10,000,000-fold for the planktonic bacteria. In contrast, an increase to 45 and then to 55° C. results in reduction of more than 200- and 1,000,000-fold with the biofilm bacteria.

Similar experiments were performed using titanium coupons (19 mm×25.4 mm×2 mm) heated in a water bath to 60° C., with similar yet less effective results. In this experiment, titanium coupons cultured with MRSA under 1×TSB containing media for 1 week at 37° C. The media was changed daily and replenished daily. Coupons were collected and washed three times in 25 ml PBS by gentle inversion and placed in a 50 ml tube which was filled with 50 ml of TSB. The samples which were to be exposed to higher temperatures had the tube and solutions heated to 60° C. The vancomycin was then added 0.3 μg/ml and 1 μg/ml when the samples were introduced to the heated sample tubes and solution, with controls receiving no vancomycin. RT controls with no vancomycin, 0.3 μg/ml, and 1 μg/ml were also performed. The supernatant of each tube was processed by removing the supernatant, pelleting, and resuspending in 1 ml of TSB. Titanium coupons were decanted into a 10 ml dish. In the center of the plate the coupons were scraped firmly in 1 motion from top to bottom avoiding the edges. The scrapers were washed in 1 ml of TSB to prepare the biofilm culture. All samples were vortexed. Samples were serially diluted and microtiters for CFU performed on TSB plates in duplicates with 10-fold dilutions starting at 0, 5 μl plated. This can be seen in FIG. 5-H. These less effective results may be attributed to the heating curve being less and not effectively inducing a significant shock or functional disruption of the biofilm on the titanium. These results may have occurred due to the titanium acting as a heat sink allowing the bacteria to adjust to the new temperature. The temperature differential may not allow for such a rapid heating curve of the micro-organisms.

These experiments highlight that treatments at temperatures as low as 45° C. can cause biofilm and planktonic bacteria to be disrupted, weakened, and made more susceptible to antibiotics. These experiments highlight that very low/low radio frequencies, which cause an even and rapid heating profile, can cause heat shock or functional disruption to microbes and biofilms which render them susceptible at low temperatures to antimicrobials. These examples demonstrate a non-invasive treatment option at low temperatures, which using temperature change with a rapid heating curve to weaken the micro-organisms renders it more susceptible to antimicrobials treatments allowing for resistant organisms to be treated. This also demonstrates that microorganisms, not just in biofilm form, but also planktonic microorganisms near the coupon, are similarly affected by this combination treatment at lower temperatures. In certain embodiments, this type of treatment may be able to be performed at even lower temperatures if the heating curve is sufficient.

One exemplary treatment method entails deploying a wire of the wire system around a fatty limb with a prosthetic which is infected, or which may become infected, as this could be used as a post-operative treatment. A patient is administered the recommended anti-biological (antibiotic, anti-fungal, etc.) and potentially a pain medication. Alternatively, the patient is sedated or remains sedated from the implantation surgery. Dosages of the antibiological will vary on based the particular biological agent, the recommended dosage range, and particulars of the patient. This methodology may allow for the recommended dosage range to be lowered. The wire system is then deployed around the area of the prosthetic and connected to the quick connect/rapid disconnects. In some embodiments of the treatment, pre-op imaging may be utilized to determine where bone cement is, the thickness of the bone cement, and where the infection is. This information is then used to determine which guides may be deployed or the orientations of the wire system. An AI system may analyze this data and select orientations of the wire system and develop an EMF treatment setting, and it may even design 3D printing guides for deploying the wire system. The guiding system may be disposable or sterilizable and reusable. In some embodiments where the bone cement that is impregnated with metal parts is used, one consistent EMF field may be utilized, while in others, the bone cement must be heated through to its outermost edges. In this scenario the EMF may need to be stronger in some areas, which can be achieved by using a separate wire, or decreasing the diameter in the selected area. The power system at this point would be adjusted to the correct EMF strength settings and exposure time selected. The cooling system is activated which may make the wire more rigid and will cool the electronics of the power system. The power system is turned on and an EMF is emitted along the unshielded portion of the wire. The resonant frequency lock alters the oscillators to converge on the resonant frequency of the wire configuration and load (the implant). This field generates an even rapid heating curve in the metal at a depth of 1-5+ mm of the prosthetic, which ensures even heating of the site of infection or potential infection and effective administration of the EMF field at a distance. The limb may be extremely fatty and to this end, the variable power system allows for a variety of different field strengths to be administered in the case of different size wires needing to be used due to a larger sized leg or the field needs to extend out further. The fatty limb may be excessively large to the point that the focal point of the field may not be on the surface of the implant. In such a case, the fringe EMF shown in FIG. 3F as 11b may be used for induction heating. In addition, the biological agent may have formed a biofilm on the bone cement, in areas where bone cement is relevant, the field strength may be increased or the proximity of the wire system to the site of treatment may be decreased, so as to create the relevant heating curve and heat transfer necessary to treat the film through the bone cement. The low radio frequency EMF may be applied continuously or in bursts in order to maintain a steady heat or create a particular heat curve. The amount of time that the EMF is applied for may vary due to the strength of the EMF, the type of biological agent, or if the treatment is used as a prophylactic treatment post-operatively. The treatment time may last a few seconds due to the rapid heating curve or may last a few minutes. The temperature that may be achieved is from about 39-70° C. The temperature may be achieved in less than a second but no greater than 7 minutes.

Another treatment method entails using a wound irrigation system such as the one featured in CN102793954B. A patient is administered the recommended anti-microbial (antibiotic, anti-fungal, etc.) and may be administered a pain medication or may be sedated. Dosage of the anti-biological will vary depending on the particular biological agent used. This methodology may allow for the recommended dosage to be lowered which may have benefits for the patient by limiting some of the side effects of the anti-biological. The temperature range in this scenario would be about 41-70° C. and can be achieved by low radio frequency EMF applied in short bursts, various strengths, or by using alternative heating sources. However, it should be noted the heating curve will be more rapid if generated with an induction system, which may translate to better results in the wound. The EMF could be powerful enough to heat the inline liquid going into the wound to 70° C. in seconds, while not heating subsequent liquid following the heated solution into the wound. This could allow for rapid heating and cooling of the wound in order to not cause damage to the tissue, but to effectively deliver the treatment to the biological agent. This treatment may be used to heat the liquid inline to at least about 41° C. for a short time then cooled. This treatment regimen may be repeated in both instances. It should be noted that this temperature is not to be continuously maintained but rather cycled or achieved once and then allowed to return to normal operating parameter, which CN102793954B describes as below 40° C.

While CN102793954B describes maintaining the wound within a temperature range, the method of the present invention involves temporary increases in temperature for the purpose of weakening a biological agent so that it becomes more susceptible to its environment, the immune system, and/or anti-biologicals. Another such method which may prove to be effective is to reduce the wound to its natural unaltered temperature which according to the patent cited can be approximately 24° C. Then as described above, cause a rapid spike in the inflowing fluid to body temperature or slightly above it, which may be sufficient in weaking the biofilm and planktonic organisms to render them susceptible to treatment. Thermocycling in this manner may be an effective means of treatment and using the wire system's built-in cooling system may be effective for cooling the incoming liquid line as well as heating it. Planktonic bacteria are not biofilms and may be harder to weaken using this methodology. In the above experiments, it was shown that a minimum of 45° C. was required to weaken planktonic bacteria.

The treatment methods disclosed herein are designed to make biological agents more susceptible to antibiologicals, the immune system, and/or their surrounding environment. EMF-induced heating may be administered in bursts, at lower temperatures between about 39-70° C. when dealing with biofilms, for variable amounts of time, with variable heat ramps, with variable strengths of EMF, or with various heating appliances and methodologies. The temperature achieved may be less for prosthetics, while wound temperatures may be higher due to planktonic microbes and/or the uneven heating nature of the wound heating apparatus. This disclosure demonstrates a non-invasive way to perform such a treatment. This experiment disclosure displays the effectiveness of such treatments and may have applications for biological treatments which extend outside the scope of the biomedical field. For instance, algae which form on surfaces (such as in the water treatment context) may be treated and made susceptible to their environment.
Example 2: Application of Induction Coil to removal of hip joint implant from bone cement.

Replacement implants are often installed using various bone cements. When an implant requires removal and replacement of the implant, the cement complicates the removal of the implant. The cement is quite hard and requires high-speed saws, chisels, and other extreme measures for removal. These procedures are technically difficult and expensive and require that the patient be anesthetized for a relatively long time. We demonstrate herein the use of an induction coil in softening temperature-sensitive bone cement to facilitate removal of the implant.

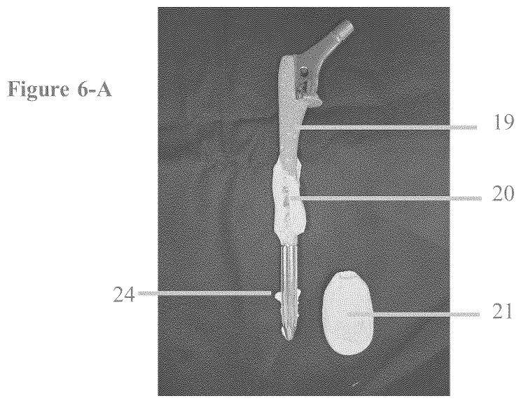

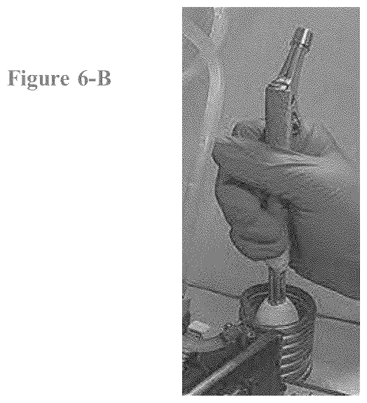

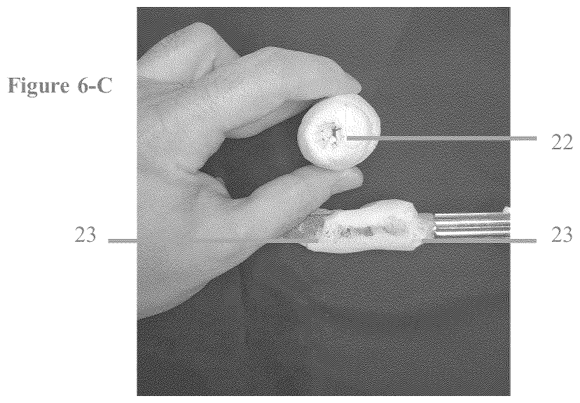

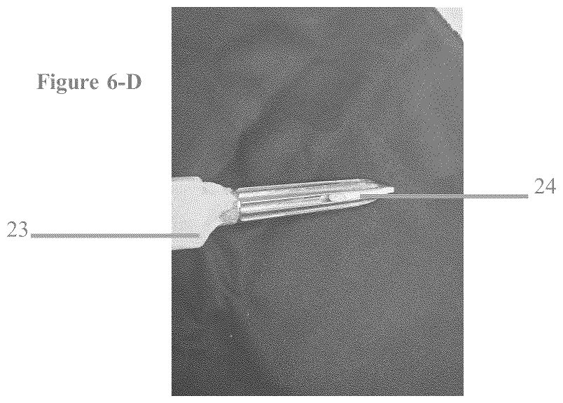

FIG. 6A shows the femoral portion of a prosthetic hip implant 19 (Smith and Nephew, Echelon revision stem made of cobalt chromium alloy) which is used for hip replacement surgeries. The lower, pointed section, is implanted into the femur while the upper section docks into the hip socket replacement implant. FIG. 6A shows the hip implant after the experiment had been performed. Simplex HV radiopaque bone cement (Benzoyl peroxide 0.74%, Poly(methylacrylate/methylmethacrylate) 82.6%, Zirconium dioxide 14.70%,) was affixed to the implant in two different places for the purpose of this study. The bone cement was prepared as per the manufacturer's instructions. The entire contents of corresponding packets of powder were mixed with the liquid in an inert mixing vessel until the material made a homogenous dough, which took 43 seconds. The powder was not prechilled. Cement was applied using surgeon's gloves and affixed to the implant. It was then allowed to cure. After the curing time, the bone cement had hardened and could not be removed from the implant. The cement was struck repeatedly with a large hammer. FIGS. 6C and 6D show the shattered remains of the middle section of cement 20 remaining on the implant 19 which show the material is brittle and not ductile at room temperature.

After shattering the cement 20, a second piece of cement was attached to the implant and allowed to cure completely from a second package of Simplex HV. FIG. 6B shows the implant 19 being held within an induction coil for the purpose of heating the implant 19 and warming the bone cement 20. The AC EMF was administered for 5.5 minutes to heat the exterior surface of the implant 19. The bone cement portion was slightly warm to the touch while the metal above and below was cool showing that the field was focused on the implant at the location of the bone cement.

After the treatment with the EMF, the bone cement 20 had become ductile enough for the implant 19 to be removed without undue effort. The bone cement 20 became plastic and deformed under tension as shown at 22 in FIG. 6C which shows clear yielding and a ductile fracture, instead of a brittle fracture which can be seen at 23. FIG. 6D shows where the bone cement seen in FIG. 6C at 22 had been attached to the implant and filled the end portion of the implant. FIGS. 6A and 6D at 24 both show plastic deformation which can be seen along the whole edge of the cement, where the material yielded significantly because it had become more ductile. This highlights the change in mechanical properties in the bone cement which is the result of the treatment/heating from the joint interface delivered from the EMF. The data described shows that through fracture mechanics, the material became more ductile when heated in this manner.

One such treatment method entails deploying a wire of the wire system around an implant revision or original implantation. Pre-operation imagery would be performed, and a technician or surgeon would determine placement and the geometry of the wire system as well as the field strength settings. An AI may determine the settings and geometry of the wire system to produce effective settings. In this case and scenario in which the technician or surgeon defines the settings, the surgeon or technician has a significant amount of input, as the surgeon is performing the surgery. For deployment, the wire would be disconnected from the power system using the quick connect/rapid disconnects and oriented around the prosthetic to be explanted. This can be done by wrapping the wire around the leg and surgical site and, due to its semi-rigid nature, will maintain its shape so as to not touch areas of interest and provide a consistent heating profile while in use. A disposable/reusable orientation guide may be utilized to orient the wire system around the limb, which may be AI generated, or prefabricated with certain geometries. The wire is then connected via the quick connect/rapid connectors to the power system. The cooling system can then be turned on and allowed to cool the wire, this may also increase the rigidity of the wire. The power systems may be adjusted based on the material of the implant or other factors. The power system will then be turned on. The EMF field may be deployed in bursts or continuously and will be used to heat the implant and bone cement. The speed of heating may be a few seconds to a few minutes. The bone cement being PMMA will become soft close to the implant and ductile. The time frame that this will occur is dependent on the strength of the EMF field and the manner it is emitted, as well as the material of the implant. Once ductile, the PMMA can be removed from the implant (pulled off directly) and in some cases, it may even be possible to simply pull the implant out of the PMMA. Once the implant is removed, the cement can be removed, drilled, or reamed (using hot metal) to create a consistent size hole in the bone cement with no debris sticking out, as some bone cement may have ripped or contorted during the process of removal. Alternatively, the bone cement may be cut out with a saw, which would take considerably less time and cause less damage as the metal implant is no longer present. Alternatively, utensils may be heated, which may be used to heat the bone cement rendering it plastic and removable, it can then be removed separately after the implant has been removed. Alternatively, yet again, the bone cement may be heated, as in some embodiments it contains metal and may be removed once it becomes plastic. New bone cement can be applied to the new implant and inserted into the cavity. This bone cement, having the same active compounds as the previous bone cement, will crosslink and form bonds with it. In the embodiments of the treatment, where the bone cement is impregnated with metal particles, after the implant is removed, the bone cement may remain in a heated ductile state, by exposing it to the field so as to help in the removal process, as opposed to cutting the bone cement.

This disclosure demonstrates one embodiment of the treatment method. This treatment plan which is designed to increase the plasticity or change other material properties of the affixing material of a prosthetic or other object may be administered in burst increments, at various temperatures, variable amounts of time, with variable heat ramps, with variable strengths of EMF, or with various heating appliances and methodologies for the purposes of removing the implant or object from the affixing material, especially in or around the human body. This experiment displays the effectiveness of such treatments on affixing materials which increase the plasticity and changes other temperature-sensitive material properties. This disclosure also demonstrates a non-invasive way to perform such a treatment.

This data demonstrates the application of the induction device wire system for the removal of implants affixed with temperature-sensitive bone cement. This data shows that an otherwise brittle material becomes plastic and more ductile due to surface heating of the cement in contact with the implant.

The application of heat may be applied for the purpose of changing the plasticity of an affixing material which then can allow for the removal of an object from the bone cement or affixing material. Such a treatment may employ heating methodology that requires a slow and stable heating curve to not burn the material. Alternatively, such a treatment may employ a rapid heating curve so as to rapidly make the material plastic for fast removal. Such a treatment may also employ intermittent exposures to a heating source. While EMFs were used in this embodiment to display the application of the invention for use on prosthetics, the application of heat to create plasticity for the purposes of removing an affixed material in the body can be accomplished by other heating means. To the skilled person in heat generating systems, other heating appliances may be used for the same application and thus this invention is not just limited to the described system which utilizes EMFs, but to any heat generation system used for this purpose. Such heating systems may include but are not limited to infrared systems, focused ultrasound, and lasers.

This data also demonstrates the application of heat for the purpose of causing a biological agent to become more susceptible to antibiotics and/or the immune system. Such a treatment may consist of intermittent exposures to the range of temperatures such as about 39-70° C. by various means for various lengths of time. However, this embodiment is a particularly useful non-invasive method which would be effective around an implant prosthesis. It should be noted that other heat generation mechanisms can be used to accomplish this, such as the liquid irrigation system referenced in CN102793954B. To the skilled person in heat generating systems, other heating appliances may be used for the same application and thus this invention is not just limited to the described system which utilizes EMFs, but to any heat generation system used for this application's purpose. Such heating systems may include but are not limited to infrared systems, focused ultrasound, irrigation systems, heating pad systems, laser systems, and bandage systems.

The inventions are not limited to the described embodiments. The skilled person understands that the features which are described in the context of the different embodiments can be combined with each other within the scope of the invention. To the skilled person, the methods of application described and proven within can be accomplished by other means of heating, however this patent is designed to cover all heating of biological agents for the purpose of increasing susceptibility of antibiotics or the immune system and treatments of heat to materials similar to bone cement to facilitate the removal of fixed objects in the body.

### Definitions

A “wire system” refers to one or more interchangeable wires, cables, cable assemblies, cable harnesses, lines, conductive ropes, conductive ribbons, or other like devices. In surgical/medical contexts, the “wire system” is sterilizable.

A “quick connect/rapid disconnect” refers to a type of connector on the terminal ends of the wire systems, which can rapidly be connected and/or disconnected from the power system. Quick connect/rapid disconnects come in many forms in the industry and commonly utilize a ball bearing locking system, threaded locking systems, clamping locking systems, threaded and non-latching systems, bayonet latching systems, snap latching systems, non-latching systems, dry break disconnects, and other similar rapid attaching and disconnecting locking mechanisms.

A “biological agent” refers to one or more microorganisms, parasites, viruses, bacteria, fungi, algae, or other infectious agents.

A “power system” refers to a system that provides AC and/or DC electricity to the wire system. This power system may be corded, or battery powered and uncorded. This power system may be designed to change the frequencies it emits, change the amount of power it emits, may be integrated with a cooling system, may contain a control system, and may be designed to operate at key frequencies for induction heating applications.

A “cooling system” refers to a system that is designed to provide the movement of some sort of coolant material through one or more different components of the invention. The coolant material may be gas or liquid. The cooling system may utilize a pump or pressurized materials.

We claim:

1. An EMF emitting device suitable for surgical use and capable of applying one or more low to very low radio frequency EMFs to a body part of a patient to generate heat on a metallic surface of an implant due to electrical resistance of an induced current, comprising:
an induction power system, wherein the power system comprises a voltage transformer and an oscillator and wherein the power system is capable of generating a low to very low radio frequency;
a wire system, wherein the wire system is capable of deforming to a desired shape on the body part of the patient and emitting a low to very low radio frequency EMF in a desired configuration;
a connector for connecting the wire system to the power system, wherein the connector comprises one or more quick connect/rapid disconnect fittings capable of exchanging the wire system with a differently sized wire system; and
a guide capable of holding the wire system in the desired shape on the body part of the patient.

2. The EMF emitting device of claim 1, wherein the power system generates a frequency from 1 kHz to 100 kHz.

3. The EMF emitting device of claim 1 further comprising a cooling system for cooling the power system.

4. The EMF emitting device of claim 1 further comprising a cooling system for cooling the wire system.

5. The EMF emitting device of claim 4, wherein the wire system comprises a cooling tube, a conductive layer, and an electrically isolating layer.

6. The EMF emitting device of claim 5 further comprising an electromagnetic shielding layer.

7. A method of using an EMF emitting device to apply a low to very low radio frequency EMF to a body part of a patient to generate heat on a metallic surface of an implant due to electrical resistance of an induced current, comprising the steps of:
applying a preformed guide to the body part of the patient;
aligning a wire system in the preformed wire guide, wherein the wire system is capable of emitting a low to very low radio frequency EMF in a desired configuration and is connected by a connector to an induction power system comprising a voltage transformer and an oscillator capable of generating a low to very low radio frequency, and wherein the connector comprises one or more quick connect/rapid disconnect fittings; and
applying the EMF to the body part of the patient to generate heat on the metallic surface of the implant.

8. The method of claim 7, wherein the EMF is from 1 kHz to 100 kHz.

9. The method of claim 7, wherein the EMF emitting device further comprises a cooling system.

10. The method of claim 7, wherein the implant is secured to the body part with bone cement and wherein the application of the EMF is sufficient to soften the bone cement to permit the removal or repositioning of the implant.

11. The method of claim 10, wherein the bone cement includes metallic components.

12. The method of claim 7, wherein the heat generated on the metallic surface of the implant is sufficient to weaken nearby biological agents.
